(19) United States
(12) Patent Application Publication
(10) Pub. No.: US 2009/0010968 A1
(43) Pub. Date: Jan. 8, 2009

(54) Spot-on formulation useful for cosmetology and dermatology

(76) Inventors: Jean-Claude Allart, Bruay Labuissiere (FR); Jean-Marie Lefevre, Amiens (FR); Jacques Peyrot, Clermont-Ferrand (FR); Jean-Paul Marty, Les Adrets de l'Esterel (FR)

Correspondence Address: BUCHANAN, INGERSOLL & ROONEY PC, POST OFFICE BOX 1404, ALEXANDRIA, VA, 22313-1404, US

(21) Appl. No.: 11/596,334

(22) PCT Filed: May 12, 2005
(86) PCT No.: PCT/FR2005/001188

§ 371 Date: Sep. 17, 2008

(30) Foreign Application Priority Data
May 12, 2004 (FR) 0405136

(51) Int. Cl. A61K 8/06; A61K 9/107; A61K 31/203; A61K 31/133; A61P 17/10; A61P 17/08; A61P 17/00; A61Q 19/00

(52) U.S. Cl. 424/401; 514/474; 514/458; 514/725

(57) ABSTRACT

The invention relates to a novel formulation of use in cosmetology and in dermatology which uses the storage capacity of the sebaceous glands.

The formulation is of the spot-on type and comprises a lipophilic active principle chosen from vitamin A, vitamin E a lipophilic agent for combating free radicals, ceramides or sphingoid bases and steroid hormones, in combination with one or more unsaturated C₄-C₂₆ fatty acids and a liquid vehicle, having sebaceous tropism.

Application to the localized topical administration of lipophilic active principles.

The present invention relates to a novel sebaceous tropism formulation for localized topical application of use in the field of cosmetology and dermatology, to its use in cosmetics and in dermatology and to the associated cosmetic treatment methods.

The sebaceous gland was for a long time regarded by many authors as a fossil gland, the only role of which was to produce sebum and to be involved in the physiopathology of acne.

In the last ten years, research has again been directed at this problem, which has made it possible to revise the initial pejorative judgment.

In the light of these studies, it has been possible to provide information on the important role which the sebaceous gland plays in skin homeostasis. The sebaceous gland is an integral part of a broader, more complex system, the pilosebaceous unit, which comprises the hair follicle, the excretion channel and the gland itself. This unit achieves its full development in the post-puberty period under the influence of the secretion of adrenal and gonadal hormones.

The sebaceous gland thus appears as a true secondary sexual organ capable not only of metabolizing a wide variety of androgens but also, by virtue of specific enzymatic equipment, of using cholesterol as substrate for the synthesis of the androgen precursors. Furthermore, its richness in 5α-reductase makes possible effective use of testosterone. Other hormonal receptors are also present (retinoid, thyroid, vitamin D receptors), which regulate its physiological activity.

Three successive events characterize it:
the holocrine secretion-production of sebum at the end of a cell cycle of 15 days, resulting in the lysis of the sebocytes,
the flowing of the sebum within the infundibulum of the sebaceous unit,
the uniform excretion at the surface of the skin and at the head hair (100 to 500 μg/cm²); a true reservoir is involved.

The sebum is composed of squalene (15%), of waxes (25%) and of triglycerides (60%) with chains of entirely specific fatty acids genetically determined in their proportions for each individual.

The novel aspect of the sebaceous gland in lipid synthesis is thus to be emphasized.

Sebum, due to its composition can be regarded as an oil in the physicochemical sense, which indicates a significant influence of the temperature on its flow.

The role of sebum is better and better known. It is involved in the organization of the lipids within the horny layer and thus participates in the barrier effect. The waxes strengthen the impermeability of the surface of the skin. The squalenes participate in UV protection, like certain cytokines which pour out at its surface. Finally, the specific lipids of the sebum, by virtue of the action of an endogenous 6-desaturase, play a role in the modulation of inflammation.

Furthermore, specific regulation of the neuropeptides (neuroendopeptidases) and a selective response to certain hormones, including retinoids, also take place in the sebum. It should also be noted that the sebum constitutes the only route for excretion of vitamin E, that, by its contribution of glycerol, it participates in skin moisturizing and, lastly, that it participates in the regulation of the ecosystem (anti-Gram-positive activity of the isomers of palmitoleic acid).

The anatomical position of the pilosebaceous unit places the follicular reservoir at the crossing of two worlds: one an internal and living world and the other as an environment carrying germs and oxygen. This explains the continual conversion of the sebum excreted from its storage region, subsequently referred to as follicular reservoir.

Furthermore, this position also makes it a favored means for excretion of certain exogenous or endogenous molecules and a source of IL-1 alpha cytokines or of neuropeptides.

Lastly, the sebaceous gland plays an important role in the homeostasis of the skin and superficial body growths and in the immune system.

In short, the sebaceous gland simultaneously exhibits properties of secretion and of synthesis, of storage and, finally, of excretion.

Numerous cosmetic and dermatological compositions for topical use have been provided in the art for ensuring the care of the skin in various indications; for example, application WO/0176538 discloses a composition for protecting the lips comprising a mixture of lipids which are chosen from ceramides, essential fatty acids and nonessential fatty acids. Patent GB 2 004 741 relates to a composition for the treatment of alopecia based on cholesterol, on fatty acids and on a phosphoaminolipid. Patent FR 2 795 960 is based on the observation that some fatty acids can have a relaxing effect on the body and it discloses, for the treatment of anxiety, a stable microemulsion based on fatty acids comprising a free carboxylic acid group which can be administered by the oral, injectable or topical route, and the like. Another emulsion based on fatty acids is disclosed in patent U.S. Pat. No. 6,361,806 but this formulation is intended to pass through the epidermis. Patent DE 4 113 346 discloses an aqueous hair composition based on phospholipids, on oils and on alcohols. However, none of the compositions described in the art is suitable for use in the properties of the sebaceous glands.

The cosmetological or dermatological treatments are often very restrictive and can then result in a phenomenon of apathy on the part of the patient when the desired improvements are judged to be too slow to emerge.

There consequently exists an increased need in the cosmetological and dermatological field to find alternative methods of administration making possible good observance on the part of the patients.

The studies carried out by the applicant company have shown that it is possible to use the storage capacity which the sebaceous gland and more particularly the follicular reservoir constitutes to release lipophilic active principles in a prolonged fashion. Thus, by virtue of the continuous excretion of its contents at the surface of the skin, it has been found that it is possible to act, by a gradual release of the active principle thus obtained, over a great variety of skin disorders. In order to achieve suitable formulations, the applicant company has demonstrated that the vehicle must be compatible with human sebum, must not exhibit any risk of modifying its physicochemical properties and must be chosen so as to be preferentially absorbed in the sebaceous gland.

### The Present Invention

The invention thus relates to a formulation for localized topical application, comprising a saturated emulsion or solution comprising one or more lipophilic, preferably lipid, active principles in a mixture of solvents comprising one or more unsaturated C₄-C₂₆ fatty acids and a polar liquid vehicle, having sebaceous tropism. The liquid vehicle preferably comprises a polar organic solvent.

### The Formulation

The presence of an unsaturated C₄-C₂₆ fatty acid makes it possible both to provide for better vectorization of the active principle, due to its compatibility with the lipids of the sebum, and to facilitate the release of the latter during the excretion phase. The unsaturated C₄-C₂₆ fatty acid can comprise from 1 to 6 unsaturations and preferably from 1 to 6 double bonds. It can, for example, be chosen from butyric acid, caproic acid, caprylic acid, capric acid, lauric acid, myristic acid, palmitic acid, palmitoleic acid, stearic acid, oleic acid, sapienic acid, ricinoleic acid, α- and γ-linoleic acid, α- and γ-linolenic acid, stearidonic acid, arachidic acid and behenic acid. These fatty acids can be used in isolation or as a mixture with one another.

Sapienic acid is naturally present in abundance in the sebaceous gland of man. It is a monounsaturated fatty acid comprising 16 carbon atoms and a single cis double bond at the 6th carbon starting from the carboxyl end. The fatty acid and the active principle are preferably dissolved in an organic solvent in order to ensure preferential absorption in the sebaceous gland.

Mention may be made, as preferred fatty acid, of sapienic acid, linolenic acid and linoleic acid. Linoleic acid, more specifically α- or γ-linoleic acid, is preferred.

The present invention relates to a formulation having sebaceous tropism which can advantageously be provided in the form of a simple solution, preferably an anhydrous solution, or of a simple emulsion or of an emulsion of quaternary type which are saturated with active principle.

When it is in the form of a simple anhydrous oily solution saturated with active principle, the vehicle is essentially composed of the fatty acid and of a polar organic solvent.

When it is in the form of an emulsion of quaternary type, typically of a microemulsion, saturated with active principle, the polar liquid vehicle is a quaternary system comprising a hydrophilic base, an oily phase, a surface-active agent and a cosurfactant. Such a microemulsion is liquid, translucent and isotropic.

In both cases, the formulation optionally comprises a crystallization inhibitor and/or an antioxidant and/or any other excipient compatible with the method of administration used according to the present invention as described in detail below.

A “formulation for localized topical application” is also commonly referred to as formulation of spot-on type; the two terminologies can be used without distinction in the context of the present invention; they mean that they are intended to treat, via a targeted application of the active principle, specific areas of the skin subject to a specific skin condition. This type of formulation, well known in the field of veterinary medicine, is characterized in that the application of a single dose, typically of just one drop, without spreading, of said formulation to the skin makes it possible, by virtue of a dispersion phenomenon, to reach a high amount of sebaceous glands of the affected area. Thus, a spot-on formulation comprising a combination of endectocidal macrocyclic lactones and 1-N-phenylpyrazoles, of use in combating parasites in the field of veterinary medicine, is disclosed in patent U.S. Pat. No. 6,426,333.

After application of a formulation of spot-on type according to the present invention, the active principle is stored in the sebaceous glands and more particularly in the follicular reservoir and then gradually released into the sebaceous flow onto the stratum corneum, according to the physiological rhythm for excretion of the sebaceous glands. More specifically, the formulation according to the present invention exhibits in particular the advantage of making possible ready incorporation of the lipophilic active principle in the follicular reservoir of the pilosebaceous unit and then of making possible not only uniform distribution of the active principle over the skin but also a release of the active principle which is spread out over time. In this way, the active principle will then be able to exert a targeted action on the skin in the stratum corneum.

The formulations according to the present invention are not targeted at passing through the skin barrier. In other words, passage into the blood is very limited.

Of course, the objectives of cosmetic and/or dermatological treatment vary according to the active principle used.

Thus, by virtue of the formulations according to the present invention, it is possible to treat, separately or simultaneously, skin conditions directly related to dysregulation of the sebaceous glands themselves or else any other skin condition, provided that there exists a lipophilic active principle for treating it. It is clearly understood, in the latter case, that use is made exclusively of the sebaceous glands as storage reservoir for slow release of the active principle in the stratum corneum. This is the case, for example, when the active principle is a ceramide or one of its precursors, as indicated below.

Thus, among the conditions related to dysregulation of the sebaceous glands, deficiencies or excesses in sebaceous secretion are found. The deficiency in sebaceous secretion can originate from the aging of the skin and can result from conditions of localized atrophies; the excess, that is to say seborrhea, can be the source of acne or of inflammatory dermatitis, such as seborrheic dermatitis.

### The Active Principles Suitable for the Formulation

Any active principle exhibiting lipophilic properties can be used and preferably a lipid active principle. Mention may in particular be made of the following lipophilic active principles, suitable for the formulation according to the present invention: any liposoluble vitamin, such as vitamin A and its derivatives, vitamin E and its derivatives; any lipophilic agent for combating free radicals; ceramides or their precursors of sphingoid base type, such as sphingosine or phytosphingosine; steroid hormones, such as estrogens or progesterone; and essential fatty acids, such as linoleic acid and linolenic acid.

The natures and roles of various active principles listed above are described below:

Vitamin A, also known under the name of retinol, is a substance widely used in cosmetology, in particular because of its major physiological function, particularly in the regulation of the proliferation and of the differentiation of a certain number of types of cells. It is known in particular that the topical application of retinol stabilizes the equilibrium of vitamin A in the skin and that this equilibrium can be detrimentally affected in particular by exposure to UV light. Thus, insufficiency of vitamin A leads in particular to an increase in the formation of wrinkles, especially in the case of overexposure to the sun (“photoaging”). Insufficiency of vitamin A also leads to a loss in the elasticity of the skin and weakens the barrier function of the skin against microorganisms. Finally, retinol is commonly employed in the treatment of acne. Advantageously, vitamin A can be used in its natural form (retinol) or in the form of its acid (retinoic acid) or also of retinyl acetate, of retinaldehyde, of β-carotene or of retinyl palmitate, in the context of the present invention.

Vitamin E and its derivatives are substances which, by virtue of their alleged properties as agent for combating free radicals (suppressors of free radicals), are widespread in cosmetology and in dermatology. Vitamin E, also known as tocopherol, can advantageously be incorporated pure in the formulation according to the present invention (natural form) or in the ester form and in particular in the form of tocopherol acetate, of tocopherol linoleate or of tocopherol succinate.

Mention may also be made, as lipophilic agent for combating free radicals, of derivatives of α-lipoic acid, andrographolides or lipophilic derivatives of vitamin C, such as esters, for example ascorbyl palmitate and stearate.

Phytosphingosine and sphingosine, which are precursors of ceramides present in human skin, have inhibiting properties with regard to protein kinase C. Furthermore, these molecules appear to be involved in the differentiation of the keratinocytes of the epidermis. It has also been observed that sphingosines present in the stratum corneum and other layers of the epidermis inhibit the growth of certain undesirable microorganisms. They thus are applied in particular in the treatment of acne, of seborrheic dermatitis and of hyperseborrhea.

The sphingoid bases used in the formulations of the present invention can be obtained by known methods from various appropriate sources, for example from natural sources or by chemical synthesis or by fermentation. The chemical synthesis processes are generally relatively expensive and do not always make it possible to obtain the sphingoid bases having the desired stereochemical configurations. The sphingoid bases can also be obtained from animal or plant tissues by extraction and purification but these methods are generally expensive and the animal sources do not always exhibit the desired bacteriological qualities.

This is why the sphingoid bases used in the invention are preferably prepared by microbial fermentation, for example from a yeast, such as Pichia ciferii, and the phytosphingosine obtained in that way exhibits the advantage of being very similar to that of animal skin. According to a preferred embodiment of the invention, use is made, as sphingoid base, of the phytosphingosine obtained from tetraacetylphytosphingosine (TAPS), derived from Pichia ciferii, by deacetylation. The deacetylation reaction can be carried out by chemical reaction, for example by hydrolysis in the presence of potassium hydroxide, or by enzymatic reaction. In order to obtain a phytosphingosine of high purity, it is preferable to carry out a purification after hydrolysis. Any purification process known in the art may be suitable. In the context of the present invention, the sphingoid base is advantageously chosen from sphingosine, sphinganine, phytosphingosine, tetracetylphytosphingosine, N-acetylphytosphingosine, phytosphingosine hydrochloride and phytosphingosine salicylate.

Steroid hormones, such as estrogens, antiandrogens or progesterone, are known for their ability to combat certain forms of seborrhea, Fox-Fordyce disease, androgenic alopecia, acne and hirsutism.

### The Components of the Formulation

The formulation can be provided either in the form of a simple oily solution or in the form of a simple or quaternary emulsion, the formulation being in both cases saturated with lipid active principle. It comprises, in addition to the fatty acid, a polar organic solvent which can be any polar organic solvent known to a person skilled in the art which is generally acceptable in a formulation for topical application. Use may in particular be made, as polar organic solvents, of acetone, ethyl acetate, methanol, ethanol, isopropanol, dimethylformamide, diethylene glycol, dichloromethane or diethylene glycol monoethyl ether (Transcutol). A solvent capable of facilitating or improving the penetration of the active principles into the skin can also advantageously be used in the compositions of the invention, for example a nonionic amphiphilic glycerol derivative, such as 1,2-O-isopropylideneglycerol (Solketal). These solvents can be supplemented by various excipients, according to the nature of the phases desired, such as C₈-C₁₀ caprylic/capric triglyceride (Estasan or Miglyol 812) or oleic acid.

In the context of the present invention, use is preferably made of Transcutol and Solketal, which are known for their high nonselective lipophilicity.

In the case of a simple solution, a volatile cosolvent can advantageously be used in combination with the main solvent, for example an alcohol, such as ethanol and glycol, in order to facilitate the drying after application and to result in supersaturation of the active principle. In the case of a simple or quaternary emulsion, the formulation comprises an oily phase and a hydrophilic phase. This cosolvent can be nonpolar and be composed, for example, of linoleic acid.

During application to the skin, on contact with the horny layer in an area comprising sebaceous glands, the difference in partition coefficients existing between the supersaturated vehicle and the stratum corneum brings about diffusion of the lipid active principle towards the sebaceous lipids and its storage in the sebaceous glands.

The anhydrous nature of the composition of the invention is favorable to its stability over time. However, in the case of the emulsions, an aqueous phase may be present but the water does not represent more than 5% by weight, with respect to the total weight of the composition.

The oily phase is formed by the lipophilic active principle dissolved in the C₄-C₂₆ unsaturated fatty acid(s), optionally supplemented by mineral oils or vegetable oils, by unsaturated polyglycosylated glycerides or by triglycerides, but also by mixtures of such compounds.

The hydrophilic phase can be formed in particular of glycol derivatives in general, such as propylene glycol, glycol ethers, polyethylene glycols or glycerol. Propylene glycol, diethylene glycol, diethylene glycol monoethyl ether and dipropylene glycol monoethyl ether are particularly preferred.

The surface-active agents for the microemulsion can be chosen from diethylene glycol monoethyl ether, dipropylene glycol monomethyl ether, polyglycosylated C₈-C₁₀ glycerides and polyglyceryl-6 dioleate.

More particularly, when the formulation according to the present invention is provided in the form of a microemulsion, it is characterized by a stable dispersion of microdroplets of the oily phase in the aqueous phase or, conversely, of microdroplets of the aqueous phase in the oily phase. The size of these microdroplets is less than 200 nm (1000 to 100,000 nm for emulsions). The interfacial film is composed of an alternation of surfactant (S) and cosurfactant (CS) molecules which, by lowering the interfacial tension, make possible the spontaneous formation of the microemulsion.

In the microemulsion, the oily phase is present in a content which can be between 2 and 15%, more particularly between 7 and 10% and preferably between 8 and 9%, with respect to the total volume of the formulation. Generally, the aqueous phase is present in a content which can be between 1 and 4%, with respect to the total volume of the formulation. The microemulsion preferably comprises from 25 to 75% of surface-active agent and from 10 to 55% of cosurfactant, with respect to the total volume of the formulation. Furthermore, the ratio of cosurfactant to surfactant is preferably between 1/7 and ½.

Thus, the cosurfactants can be chosen from short-chain alcohols, such as ethanol and glycol.

A few compounds are common to the three components discussed above, that is to say hydrophilic phase, surface-active agent and cosurfactant.

The crystallization inhibitor can prove to be highly advantageous when there exists a risk of crystallization of an active principle. It is then useful in order for the active principle easily to reach the follicular reservoir but also in order for the release phase to be facilitated, that is to say in order for the active principle to be effectively dissolved in the sebum.

The crystallization inhibitor can in particular be present in a content of 1 to 20% by weight, with respect to the total volume of the formulation, preferably of 5 to 15%. The crystallization inhibitors which can be employed in the invention include:
polyvinylpyrrolidone (PVP), poly(vinyl alcohol)s, copolymers of vinyl acetate and of vinylpyrrolidone, polyethylene glycols, benzyl alcohol, mannitol, glycerol, sorbitol, polyoxyethylenated sorbitan esters; lecithin, sodium carboxymethyl cellulose; acrylic derivatives, such as methacrylates, and others;
anionic surfactants, such as alkaline stearates, in particular sodium stearate, potassium stearate or ammonium stearate; calcium stearate, triethanolamine stearate; sodium abietate; alkyl sulfates, in particular sodium lauryl sulfate and sodium cetyl sulfate; sodium dodecylbenzenesulfonate, sodium dioctyl sulfosuccinate; fatty acids, in particular those derived from coconut oil;
cationic surfactants, such as the water-soluble quaternary ammonium salts represented by the formula N⁺R₁R₂R₃R₄, Y⁻, in which the R₁ to R₄ radicals, which are identical or different, are optionally hydroxylated hydrocarbon radicals and Y-y⁻ is an anion of a strong acid, such as halide, sulfate and sulfonate anions; cetyltrimethylammonium bromide is one of the cationic surfactants which can be employed;
amine salts of formula N⁺R′R″R′″, in which the R radicals, which are identical or different, are optionally hydroxylated hydrocarbon radicals; octadecylamine hydrochloride is one of the cationic surfactants which can be employed;
nonanionic surface-active agents, such as optionally polyoxyethylenated sorbitan esters, in particular Polysorbate 80, polyoxyethylenated alkyl ethers; polyethylene glycol stearate, polyoxyethylenated derivatives of castor oil, polyglycerol esters, polyoxyethylenated fatty alcohols, polyoxyethylenated fatty acids, copolymers of ethylene oxide and of propylene oxide;
amphoteric surfactants, such as substituted lauryl betaine compounds;
or, preferably, a mixture of at least two of the compounds listed above.

The formulation can also comprise an antioxidant provided in order to prevent any lipoperoxidation, this agent being present in particular in a content of between 0.005 and 1% (w/v), preferably between 0.01 and 0.05%.

Use may be made of any antioxidant conventional to a person skilled in the art. Mention may in particular be made of butylated hydroxyanisole (BHA), butylated hydroxytoluene (BHT), ascorbic acid, sodium metabisulfite, propyl gallate, sodium thiosulfate or a mixture of at most two of them.

The components described above, of use in preparing the liquid vehicle of the formulation according to the present invention, are well known to a person skilled in the art and can be obtained commercially or by known techniques.

The formulations according to the present invention are generally prepared by simple mixing of the components described above; advantageously, first the active principle is mixed in the organic solvent and, subsequently, the other components are added.

### The Application of the Formulation

The formulations according to the invention are extremely effective for long periods of time in the various treatments described above.

The formulations according to the present invention can be packaged in bottles with appropriate applicators. Typically, the formulation can be applied in the form of one or more drops to be distributed over the areas to be treated.

The dosage is adjusted according to the active agent used and the condition to be treated. For example, it can be between 10 and 25 drops applied to the face, in the case of retinoic acid as a 0.5% solution.

It may also be noted that the flow of the phase of excretion of the sebum depends on the temperature. This parameter can be advantageously used when it is desired to administer a lipophilic agent for combating free radicals. This is because, as the temperature has a fluidifying effect on the sebum, the agent for combating free radicals will be able to be released more rapidly when the skin is exposed to the sun.

### The Use of the Formulation

The invention relates to a method for the cosmetic treatment of the skin intended for the treatment or prevention of damage to the skin associated with the aging of the skin which can consist of a loss in the firmness of the skin or in the tonicity of the skin and the appearance of fine lines and wrinkles, consisting in applying, to the affected areas of the skin, a formulation according to the present invention comprising vitamin A, for example in the form of retinyl acetate, of retinyl palmitate, of retinoic acid, of retinaldehyde or of β-carotene.

The invention also relates to a method for the cosmetic treatment of the skin intended for the treatment or prevention of damage to the skin associated with photoaging of the skin, consisting in applying, to the affected areas of the skin, a formulation according to the present invention comprising vitamin A, for example in the form of retinyl acetate, of retinyl palmitate, of retinoic acid, of retinaldehyde or of β-carotene, vitamin E, for example in the form of tocopherol acetate or of tocopherol succinate, or also any lipophilic agent for combating free radicals, for example in the form of α-lipoic acid, of andrographolides or vitamin C esters, such as ascorbyl palmitate and stearate.

Furthermore, the invention relates to a method for the cosmetic treatment of the skin intended for the peeling of the latter, consisting in applying, to the skin, a formulation according to the present invention comprising concentrated retinoic acid, preferably between 0.1 and 1%.

In addition, the invention relates to a formulation of spot-on type according to the present invention for its use as dermatological composition in human medicine.

More particularly, the invention relates to:
a formulation of spot-on type according to the present invention, comprising vitamin A, a steroid hormone, a ceramide or one of its precursors of sphingoid type or also an essential fatty acid, for its use as dermatological composition intended to treat acne,
a formulation of spot-on type according to the present invention, comprising a ceramide or one of its precursors of sphingoid base type or else a steroid hormone, for its use as dermatological composition intended to treat seborrhea,
a formulation of spot-on type according to the present invention, comprising a ceramide or one of its precursors of sphingoid base type or else an essential fatty acid, for its use as dermatological composition intended to treat seborrheic dermatitis,
a formulation of spot-on type according to the present invention, comprising vitamin E, vitamin A or else glycerol, for its use as dermatological composition intended to treat deficiency of sebum,
a formulation of spot-on type according to the present invention, comprising a steroid hormone with an antiinflammatory purpose, for its use as dermatological composition intended to treat Fox-Fordyce disease, or an antiandrogenic hormonal substance in the context of androgenic alopecia and of hirsutism,
a formulation of spot-on type according to the present invention, comprising a ceramide or one of its precursors of sphingoid base type, for its use as dermatological composition intended to treat chronic folliculitis of the scalp,
a formulation of spot-on type according to the present invention, comprising an essential fatty acid, for its use as dermatological composition intended to treat ichthyosis when located on the scalp,
a formulation of spot-on type according to the present invention, comprising a kojic acid ester, a licorice extract, a steroid hormone, vitamin A or else a ceramide or one of its precursors of sphingoid base type, for its use as dermatological composition intended to treat actinic lentigo, pigment disorders related to aging and hypermelanosis.

Furthermore, the invention relates to the use of a lipophilic active principle aimed at the skin for the preparation of a formulation for localized topical application in man intended to be absorbed by the sebaceous glands and then gradually released over time by the latter over the stratum corneum, according to its secretory flow.

The invention relates to the use of an active principle chosen from vitamin A, for example in the pure form (retinol) or in the form of retinyl acetate, of retinyl palmitate, of retinoic acid, of retinaldehyde or β-carotene; steroid hormones, for example estrogens, progesterone and antiandrogens; ceramides or their precursors, such as sphingoid bases, for example sphingosine, sphinganine, phytosphingosine, tetraacetylphytosphingosine, N-acetylphytosphingosine, phytosphingosine hydrochloride and phytosphingosine salicylate; essential fatty acids, for example linoleic acid and linolenic acid, for the preparation of a formulation according to the present invention intended to treat acne.

Furthermore, the invention relates to the use of an active principle chosen from ceramides or their precursors, such as sphingoid bases, for example sphingosine, sphinganine, phytosphingosine, tetraacetylphytosphingosine, N-acetylphytosphingosine, phytosphingosine hydrochloride and phytosphingosine salicylate; steroid hormones, for example estrogens, progesterone or antiandrogens, for the preparation of a formulation according to the present invention intended to treat seborrhea.

The invention also relates to the use of an active principle chosen from ceramides or their precursors, such as a sphingoid base, for example sphingosine, sphinganine, phytosphingosine, tetracetylphytosphingosine, N-acetylphytosphingosine, phytosphingosine hydrochloride and phytosphingosine salicylate; essential fatty acids, for example linoleic acid and linolenic acid, for the preparation of a formulation according to the present invention intended to treat seborrheic dermatitis.

In addition, the invention relates to the use of an active principle chosen from vitamin E, for example tocopherol acetate, tocopherol succinate; vitamin A, for example retinyl acetate, retinyl palmitate, retinoic acid, retinaldehyde or β-carotene; and glycerol, for the preparation of a formulation according to the present invention intended to treat a deficiency of sebum.

Another subject matter of the invention is the use of an active principle chosen from steroid hormones, for example estrogens, progesterone or antiandrogens, such as cyproterone or finasteride, for the preparation of a formulation according to the present invention intended to treat Fox-Fordyce disease, androgenic alopecia or hirsutism.

Finally, a subject matter of the invention is the use of an active principle chosen from ceramides or their precursors, such as a sphingoid base, for example sphingosine, sphinganine, phytosphingosine, tetracetylphytosphingosine, N-acetylphytosphingosine, phytosphingosine hydrochloride and phytosphingosine salicylate, for the preparation of a formulation according to the present invention intended to treat chronic folliculitis of the scalp.

A subject matter of the invention is the use of an active principle chosen from essential fatty acids, for example linoleic acid and linolenic acid, for the preparation of a formulation according to the present invention intended to treat ichthyosis located on the scalp.

Lastly, a subject matter of the invention is the use of an active principle chosen from kojic acid esters, for example kojic acid dipalmitate; licorice extracts (glabridin); steroid hormones, for example estrogens, progesterone and anti-androgens; vitamin A, for example retinyl acetate, retinyl palmitate, retinoic acid, retinaldehyde and β-carotene; ceramides or their precursors, such as a sphingoid base, for example sphingosine, sphinganine, phytosphingosine, tetraacetylphytosphingosine, N-acetylphytosphingosine, phytosphingosine hydrochloride and phytosphingosine salicylate, intended to treat actinic lentigo, pigment disorders related to aging and hypermelanosis.

The examples which follow illustrate the present invention without, however, limiting the scope thereof.

### EXAMPLE 1

### Study Protocol and Result (10 Cases) for Vitamin A

The purpose of this first example is to illustrate the relevance of the concept of administering lipophilic active principles in follicular reservoirs prior to their prolonged release, according to the physiological cycle.

In order to assert this concept:
the rate of absorption of the active principle after local application was determined,
its preferential storage in the pilosebaceous unit was confirmed,
its gradual and continuous release and its duration were monitored.

A novel methodology was used. A certain number of fluorescent lipids compatible with the sebaceous lipids was selected.

The skin was exposed to a Wood's lamp. The dispersion surface area after application of a single dose of the solution to be studied (cracking effect) was thus determined. Measurements of rate of absorption by the sebaceous gland were also carried out.

The reality of the release was demonstrated by simple fluorescence according to a specific protocol for each active principle under consideration. A more quantitative approach could also be introduced by virtue of the Sebutest, which is intended to measure the sebaceous flow and to collect the sebum for analysis. These parameters make it possible to foresee the duration of action of the active principle and consequently the dosage to be recommended.

Account has also been taken of the variations in the number of sebaceous glands according to the topography by carrying out the studies on different sites. This has made it possible to adjust the dosage as a function of the site of application. Likewise, the response according to age was evaluated.

### Implementation:

A comparative study was carried out on two formulations (a control formulation and a formulation according to the invention)

### Preparation:

| | | |
|---|---|---|
| Vitamin A palmitate | 3,000,000 | IU |
| BHT | 0.10 | g |
| PVP | 4 | g |
| BHA | 0.20 | g |
| Sodium fluorescein | 0.20 | g (or Nile red 0.20 g) |
| Linoleic acid | 5 | g |
| Diethylene glycol | q.s. 100 | g |

### Control:

| | | |
|---|---|---|
| Sodium fluorescein | 0.20 | g |
| 60° alcohol | q.s. 100 | g |

### Choice of the Sites of Application:

Region rich in sebaceous glands: mentolabial fold, nasogenian fold
Less rich region: forearm (front face)
Hair region: scalp

### Protocol:

A comparative study was carried out on symmetrical areas. One drop of 0.4 ml of product to be studied was deposited on each area selected. Natural drying was carried out without spreading. The following was studied:
the dispersion, by measuring the surface area covered after 15 minutes,
the absorption, by degree of fluorescence at 60 minutes and at 6 hours,
the persistence, by measuring the fluorescence at 24 hours, 48 hours and 96 hours.

At the 168th hour, the skin or the scalp was washed normally, while avoiding, however, excessively powerful surfactants. A surgras cleansing bar and a mild shampoo were recommended.

The extinction of the fluorescence was also studied.

The active principle was demonstrated by stripping of the epidermis and specific coloring with antimony trichloride, which gives a blue coloring by microscopy at 24 hours and 96 hours. Finally, the absorption spectrum (characteristic band at 328 nanometers) were studied.

### Results:

The results are identical in all the subjects.

The dispersion is better over the hair areas and over the areas rich in sebaceous glands. It is, in all the cases, superior to the control.

The absorption can be identified from the first hour with perforations on the dispersion stain corresponding to the pilosebaceous orifices.

These perforations are not encountered in the control. This aspect becomes more marked at the 6th hour, whereas the fluorescence of the control stain decreases.

The persistence is confirmed by a marked continued existence of the fluorescence, which is still observed at the 168th hour. The control stain is observed to completely disappear; the persistence is greater on the areas rich in sebaceous glands.

Consequently, the presence of vitamin A was confirmed on the 4th day on all the areas and on the 7th day on the hair areas.

The extinction of the fluorescence is greater than a period of time of 9 days.

### EXAMPLE 2

### Formulation of Retinoic Acid in a Formulation According to the Invention

| | | |
|---|---|---|
| Retinoic acid | 0.30 | g |
| BHT | 0.10 | g |
| BHA | 0.20 | g |
| Linoleic acid | 5.00 | g |
| PVP | 4.00 | g |
| Transcutol | q.s. 100 | g |

### Study of Effectiveness and of Tolerance Versus 0.1% Locacid® Solution

5 patients received the spot-on formulation and the other 5 received Locacid®. The product was applied to the forehead, the cheeks, the wings of the nose and the chin: 1 single application for the spot-on formulation and 1 daily application for the Locacid®.

The observations were carried out on D1, D2, D3, D4, D5, D6 and D8.

The skin types were identical, namely normal skin with a greasy tendency; the patients were aged from 35 to 50 years.

In the patients treated with the Locacid®, irritating phenomena are observed from D2, with redness, burning sensation and dryness. It was necessary to halt the treatment after the 3rd application in 4 cases out of 5 and on the 4th day for the 5th patient.

In the patients treated with the spot-on formulation, a progressive activity is observed which is free of irritating phenomena and which is visible in the form of a fine desquamation, which begins on the 3rd day and continues up to the 6th day, and an overall improvement in the skin surface.

This study demonstrates that the spot-on formulation of the invention is superior to Locacid®, that the action is progressive and that it extends over 8 days under conditions of noteworthy tolerance, making it possible to be able to judge the true effects of the product.

These observations make it possible to provide a novel method of administration of vitamin A acid (retinoic acid) which is much more effective than the available known proposals.

The ease of treatment with a single application per week is a guarantee of observance.

### EXAMPLE 3

### Other Examples of Formulations According to the Invention and Results after Treatment

Other examples were carried out in order to illustrate the variety of treatments which can be envisaged by virtue of the formulations according to the present invention.

### Spot-On Based on Linoleic Acid

2 applications per week to treat retentional acne.

Aim: correcting deficiencies in linoleic acid in the sebum of patients suffering from acne.

The results proved to be very positive.

### Spot-On Based on Phytosphingosine and on Linoleic Acid Versus Ketoconazole and Local Corticoids in Taking Care of Seborrheic Dermatitis

The formulation used was as follows:

| | | |
|---|---|---|
| Salicyloyl phytosphingosine | 0.5 | g |
| Tocopherol | 0.5 | g |
| Caprylic/capric C₈-C₁₀ triglyceride | 10.0 | ml |
| Ethanol | 13.5 | ml |
| PVP | 5.0 | g |
| Tween 80 | 5.0 | g |
| Transcutol | 60 | ml |
| Propylene glycol | q.s. 100 | ml |

2 applications per week against one application per day of ketoconazole and local corticoids.

The results proved to be very positive. This is because the effectiveness of the spot-on formulation was faster than that based on ketoconazole and local corticoids. Thus, the weaning from corticoids could be facilitated. Furthermore, reoccurrence is not observed on halting the treatment.

### Spot-On Based on Phytosphingosine and on Linolenic Acid in Chronic Folliculitis of the Scalp

The formulation used was as follows:

| | | |
|---|---|---|
| Phytosphingosine hydrochloride | 0.4 | g |
| γ-Linolenic acid | 5.0 | g |
| Ethanol | 20 | ml |
| Transcutol | q.s. 100 | ml |

2 applications per week.

A result could be observed from the 2nd week of treatment.

### Spot-On Based on Licorice Extract and on Linoleic Acid in the Treatment of Actinic Lentigo

The formulation used was as follows:

| | | |
|---|---|---|
| PT40 (licorice extract) | 0.80 | g |
| Linoleic acid | 5.0 | ml |
| Polyglyceryl dioleate | 4.5 | ml |
| Ethanol | 15.0 | ml |
| Transcutol | 60.0 | ml |
| Tween 80 | 50.0 | g |
| PVP | 5.0 | g |
| Propylene glycol | q.s. 100 | ml |

1 application every 5 days in the treatment of actinic lentigo of the face.

A significant lightening is observed after 4 weeks of treatment.

### Retinoic Acid+Palmitic Acid Spot-On in Peeling Versus AHA or Trichloroacetic Acid

The formulation used was as follows:

| | | |
|---|---|---|
| Retinoic acid | 0.5 | g |
| Palmitic acid | 4.0 | g |
| PVP | 5.0 | g |
| BHT | 0.1 | g |
| BHA | 0.2 | g |
| Transcutol | q.s. 100 | ml |

Better results were observed than with α-hydroxyl acids (AHA) and equivalents comprising 30% trichloroacetic acid without social embarrassment or pigment trouble postpeeling.

### Tocopherol+Palmitoleic Acid Spot-On in Antisun Protection

The formulation used was as follows:

| | | |
|---|---|---|
| α-tocopherol | 1.0 | g |
| Palmitoleic acid | 5.0 | g |
| Tween 80 | 50.0 | g |
| PVP | 5.0 | g |
| Ethanol | 10 | ml |
| Transcutol | q.s. 100 | ml |

1 application per week.

A strengthening in natural photoprotection is observed.

### Linoleic Acid Spot-On in Ichthyosis of the Scalp

2 applications per week.

| | | |
|---|---|---|
| Linoleic acid | 5.0 | g |
| Tocopherol | 0.5 | g |
| BHT | 0.1 | g |
| BHA | 0.2 | g |
| Tween 80 | 10.0 | g |
| Isopropanol | 40 | ml |
| Diethylene glycol | q.s. 100 | ml |

A normalization of the desquamative state is observed.

1. A formulation of spot-on type for localized topical application, comprising a saturated emulsion or solution comprising a lipophilic active principle in a mixture of solvents comprising one or more unsaturated C₄-C₂₆ fatty acids and a polar liquid vehicle, having sebaceous tropism.

2. The formulation of spot-on type as claimed in claim 1, wherein the active principle is selected from the group consisting of vitamin A; vitamin E; an lipophilic agent for combating free radicals; ceramides or sphingoid bases; and steroid hormones.

3. The formulation of spot-on type as claimed in claim 2, wherein vitamin A is provided in natural form (retinol), in the form of its acid (retinoic acid) or also of retinyl acetate, of retinaldehyde, of β-carotene or of retinyl palmitate; vitamin E is provided in the pure form or in the form of an ester, such as tocopherol acetate, tocopherol linoleate or tocopherol succinate; the lipophilic agent for combating free radicals is chosen from derivatives of α-lipoic acid, andrographolides or lipophilic derivatives of vitamin C, such as ascorbyl palmitate and stearate; the sphingoid base is chosen from sphingosine, sphinganine, phytosphingosine, tetracetylphytosphingosine, N-acetylphytosphingosine, phytosphingosine hydrochloride and phytosphingosine salicylate; and the steroid hormones are chosen from estrogens, antiandrogens and progesterone.

4. The formulation of spot-on type as claimed in claim 1, wherein the unsaturated C₄-C₂₆ fatty acid is selected from the group consisting of sapienic acid, linolenic acid and linoleic acid.

5. The formulation of spot-on type as claimed in claim 4, wherein the unsaturated C₄-C₂₆ fatty acid is α- or γ-linoleic acid.

6. The formulation of spot-on type as claimed in claim 1, wherein it is provided in the form of a simple oily solution, of an emulsion or of an emulsion of quaternary type which are saturated with active principle.

7. The formulation of spot-on type as claimed in claim 1, wherein the formulation is provided in the form of an anhydrous simple oily solution saturated with active principle and in that the polar liquid vehicle comprises an organic solvent.

8. The formulation of spot-on type as claimed in claim 7, wherein the organic solvent is selected from the group consisting of acetone, ethyl acetate, methanol, ethanol, isopropanol, dimethylformamide, diethylene glycol, dichloromethane, 1,2-O-isopropylideneglycerol (Solketal) and diethylene glycol monoethyl ether (Transcutol).

9. The formulation of spot-on type as claimed in claim 1, for its use as dermatological composition in human medicine.

10. The use of a lipophilic active principle aimed at the skin for the preparation of a formulation for localized topical application in man intended to be absorbed by the sebaceous glands and then gradually released in the sebaceous flow over the stratum corneum, according to the physiological rhythm for excretion of the sebaceous glands.

11. The use of a lipid active principle chosen from vitamin A, steroid hormones, ceramides or their precursors, such as sphingoid bases, and essential fatty acids for the preparation of a formulation of spot-on type intended to treat acne.

12. The use of a lipid active principle chosen from ceramides or their precursors, such as sphingoid bases, and steroid hormones for the preparation of a formulation of spot-on type intended to treat seborrhea.

13. The use of a lipid active principle chosen from ceramides or their precursors, such as a sphingoid base, and essential fatty acids for the preparation of a formulation of spot-on type intended to treat seborrheic dermatitis.

14. The use of a lipid active principle chosen from vitamin E, vitamin A and glycerol for the preparation of a formulation of spot-on type intended to treat a deficiency of sebum.

15. The use of a lipid active principle chosen from steroid hormones for the preparation of a formulation of spot-on type intended to treat Fox-Fordyce disease, androgenic alopecia or hirsutism.

16. The use of a lipid active principle chosen from ceramides or their precursors, such as a sphingoid base, for the preparation of a formulation of spot-on type intended to treat chronic folliculitis of the scalp.

17. The use of a lipid active principle chosen from essential fatty acids for the preparation of a formulation of spot-on type intended to treat ichthyosis of the scalp.

18. The use of a lipid active principle chosen from kojic acid esters, a licorice extract, steroid hormones, vitamin A, ceramides or their precursors, such as a sphingoid base, for the preparation of a formulation of spot-on type intended to treat actinic lentigo, pigment disorders relating to aging and hypermelanosis.

19. (canceled)

20. A method for the cosmetic treatment of the skin, comprising applying, to the affected areas of the skin, a formulation of spot-on type as claimed in claim 1.
